(19) United States
(12) Patent Application Publication
(10) Pub. No.: US 2019/0247142 A1
(43) Pub. Date: Aug. 15, 2019

(54) IMAGE PROCESSING METHOD AND APPARATUS USING ELASTIC MAPPING OF VASCULAR PLEXUS STRUCTURES

(71) Applicant: LEICA INSTRUMENTS (SINGAPORE) PTE. LTD., Signapore (SG)

(72) Inventor: George THEMELIS, Lindau (DE)

(21) Appl. No.: 16/269,968

(22) Filed: Feb. 7, 2019

(30) Foreign Application Priority Data
Feb. 15, 2018 (EP) 18156906.2

(51) Int. Cl. A61B 90/00; G06T 3/00; G06K 9/62; G06T 7/30; A61B 5/026; A61B 90/20; A61B 1/04

(52) U.S. Cl. CPC A61B 90/37; A61B 2505/05; G06K 9/6201; G06T 7/30; A61B 5/0261; A61B 90/361; A61B 90/20; A61B 1/043; G06K 2209/05; G06T 2207/30104; G06T 2207/20221; G06T 2207/10056; G06T 2207/10068; G06T 2207/10048; A61B 2090/367; A61B 2090/373; A61B 2090/365; A61B 2090/3941; A61B 2090/3979; G06T 3/0081

(57) ABSTRACT

The invention relates to an image processing method and a medical observation device (1) such as a microscope (2) or endoscope. The device and method are used for displaying output image data (54) of soft biological tissue (12). In image-guided surgery, pre-operative three-dimensional image data (6) of the soft biological tissue (12) are elastically matched to interoperative image data (14) which are acquired during surgery. By displaying the elastically matched pre-operative three-dimensional image data (6) together with the interoperative image data (14), the surgeon may be made aware of the consistence of the soft biological tissue (12) below the visible surface layer. Existing systems for image-guided surgery need to be manually readjusted if surgery is done on soft biological tissue (12), which may deform and shift. To avoid this, the device and method according to the invention perform an elastic matching of the pre-operative three-dimensional image data (6) based on the interoperative image data (14) of the soft biological tissue (12). At least one vascular plexus structure (48) is first identified in the interoperative image data (14) and then the same vascular plexus structure (48) is identified in the pre-operative three-dimensional image data (6). The vascular plexus structure (48) in the pre-operative three-dimensional image data (6) is then elastically matched to the vascular plexus structure (48) in the interoperative image data (14). Output image data (54) are generated combining the elastically matched pre-operative three-dimensional image data (6) to the interoperative image data (14). Preferably, the at least one vascular plexus structure (48) is identified using fluorescent light from a fluorophore (22) which has been injected into the soft biological tissue (12).

### CROSS REFERENCE TO RELATED APPLICATIONS

This application claims priority of European patent application number 18156906.2 filed Feb. 15, 2018, the entire disclosure of which is incorporated by reference herein.

### FIELD OF THE INVENTION

The invention relates to an image processing method and a medical observation device for displaying soft tissue images, in particular in real time during surgery.

### BACKGROUND OF THE INVENTION

Image-guided surgery is nowadays commonly used for certain kind of surgical operations, such as brain surgery. Image-guided surgery uses stored pre-operative three-dimensional information in the form of image data about the operation area. Such pre-operative three-dimensional image data may, for example, have been obtained using magnetic resonance imaging. During surgery, the pre-operative information is visually aligned with the actual optical view of the tissue to be operated on. Using the pre-operative three-dimensional information, tissue structures, such as tumors or vascular plexus structures, may be visualized that are otherwise invisible under the visible tissue surface.

Use of the pre-operative three-dimensional information helps the surgeon to find and reach a certain area of the tissue, to avoid sensitive tissue such as nerves, arteries and veins, and/or to remove certain tissue effectively, such as a tumor.

For the alignment of the pre-operative three-dimensional information to the actual view of the surgeon, either stereo infrared cameras or sensors for detecting optical or electromagnetic markers fixed on the patient's body are typically used.

The spatial accuracy of these methods is not sufficient, however, if surgery is performed on soft tissue, which can shift and deform relative to the markers.

Therefore, in existing systems, at one point during surgery, the surgeon needs to align the pre-operative three-dimensional information manually with his actual view. This consumes time and time is critical for all surgery. Moreover, the surgeon is distracted from the surgery, which is also to be avoided.

### SUMMARY OF THE INVENTION

It is therefore the object of the invention to provide a method and device which allow the performance of image-guided surgery even if the surgery is performed on soft tissue which may easily deform and shift during the operation.

According to the inventive method, this object is solved by an image processing method for displaying soft tissue images, in particular in real time during surgery, which comprises the steps of: providing pre-operative three-dimensional image data of soft biological tissue; acquiring interoperative image data of the soft biological tissue in at least one of the visible-light spectrum and the near-infrared spectrum; automatically identifying at least one vascular plexus structure in the interoperative image data; identifying the at least one identified vascular plexus structure in the pre-operative three-dimensional image data; elastic matching of the pre-operative three-dimensional image data to the interoperative image data by mapping at least part of the pre-operative three-dimensional image data to the at least one identified vascular plexus structure in the interoperative image data, forming output image data from the elastically matched pre-operative three-dimensional image data and the interoperative image data.

To solve the above problem, the medical observation device for the observation of soft tissue images, in particular during surgery, comprises a memory assembly comprising pre-operative three-dimensional image data; a camera assembly for acquiring interoperative image data in at least one of the visible-light spectrum and the near-infrared spectrum; an image processor assembly, which comprises a pattern-matching module for identifying at least one vascular plexus structure in the interoperative image data and for identifying the at least one identified vascular plexus structure in the pre-operative three-dimensional image data, a matching module for elastically matching the pre-operative three-dimensional image data to the interoperative image data based on the at least one identified vascular plexus structure, and an image-forming module for combining the elastically matched pre-operative three-dimensional image data with the interoperative image data to the output image data; and an output interface for outputting the output image data.

The method and device according to the invention allow the continuous performance of image-guided surgery in soft tissue even if the tissue deforms and moves within the body without the need to manually realign the pre-operative image data to the interoperative image data. This is because the structure which is used for elastically matching the pre-operative three-dimensional image data is a structure which is part of the soft tissue and thus deforms and moves together with the soft tissue. The pre-operative three-dimensional image data are thus mapped continuously to what the surgeon actually sees. In fact, the visual information that is available to the surgeon is itself used to align the pre-operative three-dimensional image data.

The image processing method and the medical observation device according to the invention may be improved by adding one or more of the following features. Each of the following features can be added independently of the remaining features. Each of the following features has its own advantageous technical effect. Further, the following features can all be added equally to both the method and the device.

The modules described above can be implemented in software, hardware or a combination of both software and hardware. Further, the differences between the particular modules are primarily functional. Different modules may thus be comprised of a single or a plurality of electric components and/or a single logical unit, such as a single sub-routine.

The output image data may be displayed on a display assembly, which may be part of the medical observation device.

The step of elastic matching may include a technique as is described in Gee, J. C.; Reivich, M.; and Bajcsy, R.: “Elastically Deforming a Three-Dimensional Atlas to Match Anatomical Brain Images” (1993). IRCS Technical Reports Series. 192. The step of identifying a vascular plexus structure may use the method as described in Suri, J. S.; Laxminarayan, S. [eds]: “Angiography and Plaque Imaging: Advanced Segmentation Techniques”, CRC Press, 2003, pp. 501-518, and Rouchdy, Y.; Cohen, L.: “A Geodesic Voting Method of Tubular Tree and Centrelines”, DOI: 10.1109/ISBI.2011.5872566, 2011, pp. 979-983. The matching module may in particular be configured to execute a matching routine described in any of these references.

In one embodiment, the identification of the at least one vascular plexus structure may be done preferably exclusively with interoperative fluorescent-light image data. Such fluorescent-light image data may be obtained by injecting a fluorophore, such as indocyanine green, into the tissue. The camera assembly may comprise an optical filter assembly, such as a band-pass filter assembly, of which the pass band is restricted to the fluorescence spectrum of the fluorophore. As the fluorophore is transported by the blood, the vascular plexus structure may be more easily defined in the fluorescent-light image. The medical observation device may have an illumination assembly, having an illumination spectrum which comprises light in wavelengths that trigger fluorescence of the fluorophore. The illumination spectrum may be restricted to these fluorescence-triggering wavelengths.

The interoperative image data may, in another embodiment, contain both white-light image data and fluorescent-light image data. The white-light image data may be used to present output image data to the surgeon that represent what he would see with his own eyes. For this, the illumination spectrum may also comprise wavelengths of the visible spectrum, in particular white light.

The medical observation device may be one of a microscope and an endoscope.

The interoperative image data may be two-dimensional, three-dimensional or multi-dimensional data. Three-dimensional interoperative image data may, for example, be acquired by a microscope using z-stacking or a stereoscopic setup, or a SCAPE or SPIM microscope.

The interoperative image data may be recorded simultaneously in more than three different wave bands using, for example, more than one camera, a multi-spectral camera and/or a hyper-spectral camera.

The elastic mapping may include or consist of the step of elastically matching the at least one identified vascular plexus structure in the pre-operative three-dimensional image data to the corresponding at least one identified vascular plexus structure in the interoperative image data. The mapping used for the at least one vascular plexus structure may be used for the rest of the pre-operative three-dimensional image data.

The accuracy and reliability of the mapping of the pre-operative three-dimensional image data to the interoperative image data depends on how accurately the at least one vascular plexus structure may be recognised. As already stated above, pattern recognition may be performed on the fluorescent-light interoperative image data, using regions in which the fluorophore is located and which therefore have high fluorescent-light intensity.

The method in another embodiment may comprise the step of identifying at least one arterial vascular plexus structure within the interoperative image data, and/or at least one venous vascular plexus structure within the interoperative image data. For identifying the type of vascular plexus structure and for generating output image data in which the different types of vascular plexus structures are marked in different false colors, the system and method described in European patent application EP 17 174 047.5 may be used. This application is incorporated in its entirety by reference.

Additionally or alternatively, the at least one vascular plexus structure may be identified using interoperative image data which have been acquired in at least one of a plurality of separate wavelengths e.g. by a multispectral and/or hyperspectral camera. The multi- and/or hyperspectral interoperative image data may be unmixed and/or processed to show the distribution of at least one of arterial or venous blood, or the respective blood vessels. A method and apparatus for identifying the at least one vascular plexus structure is described e.g. in Matthew B. Bouchard, Brenda R. Chen, Sean A. Burgess, and Elizabeth M. C. Hillman, “Ultra-fast multispectral optical imaging of cortical oxygenation, blood flow, and intracellular calcium dynamics,” Opt. Express 17, 15670-15678 (2009).

Another measure for facilitating the identification of the at least one vascular plexus structure is to use at least one optical cross-polarizing filter assembly for reducing specular reflections. This method is described in European patent application EP 16 204 933.2, which is herewith incorporated by reference in its entirety.

A further step towards a more reliable identification of the at least one vascular plexus structure may be to compute, at at least one location in the interoperative image data, the blood flow direction using the fluorescent-light image data. The blood flow direction can be computed at a given location by determining at least one of the temporal and derivative and the spatial derivative, as is described in European patent application EP 17 210 909.2, which is herewith incorporated by reference in its entirety. A location of the interoperative image data may respond to a single pixel or a coherent array of pixels. Additionally or alternatively, a principal component analysis may be used to determine blood flow direction as description in European patent application EP 17 174 047.5, which is also included by reference in its entirety.

The mapping of the pre-operative three-dimensional image data to the interoperative image data may be facilitated if in addition to the mere image data, additional information about the position of the interoperative image data, and thus the vascular plexus structure within the tissue, is available. For this, a position sensor may be provided for generating position data representative of the position of a field of view of the camera assembly. The position data may comprise at least one of focal length, field depth and distance setting of the camera assembly. The position data may comprise at least one of incremental position data and absolute position data. Incremental position data may be used to indicate the change of the position of the field of view from one frame of interoperative image data to a subsequent frame of interoperative image data. Absolute position data may be used for each frame of the interoperative image data to indicate absolute position with reference to a constant reference. Changes of the position data between subsequent frames of interoperative image data may then be obtained by computing differences of the respective absolute position data. The position data may, in one embodiment, be used in at least one of identifying the at least one vascular plexus structure in the three-dimensional data, elastically matching the pre-operative three-dimensional image data to the interoperative image data, and displaying the output image data.

Finally, the invention is also directed to a non-transitory computer-readable medium storing a programme causing a computer to execute the method in any of the above-described embodiments.

In the following, an embodiment of the invention is exemplarily described with reference to the accompanying drawing. The combination of features shown in the exemplary embodiment is for explanatory purposes only. A feature can be omitted from the embodiment if the technical effect of the respective feature as described above is not needed for a particular application. Conversely, one or more of the above-described features may be added to the embodiment if the technical effect(s) of the one or more technical feature(s) is needed for a particular embodiment.

### BRIEF DESCRIPTION OF THE DRAWING VIEW

FIG. 1 shows an exemplary embodiment of the method and device according to the invention.

### DETAILED DESCRIPTION OF THE INVENTION

The configuration and function of an optical observation device 1 for observing live tissue, in particular during surgery, is explained. The medical observation device is shown to be a microscope 2 just for the purposes of explanation. The medical observation device 1 may also be an endoscope (not shown).

The medical observation device 1 comprises a memory assembly 4, in which pre-operative three-dimensional image data 6 are stored. The memory assembly 4 may comprise standard computer memory.

The medical observation device 1 further comprises a camera assembly 8, which has a field of view 10. During surgery, soft biological tissue 12, such as brain tissue, muscle tissue, lymph tissue or tissue of an internal organ or of other soft body parts, may be arranged in the field of view 10. During surgery, the camera assembly 8 acquires interoperative image data 14, which may be structured as a single input frame 16 or a time series 18 of input frames 16. The interoperative image data 14 may comprise or consist of pixels 20. The interoperative image data 14 may be two-dimensional, i.e. representing a plane in the field of view 10, three-dimensional, i.e. representing a volume in the field of view 10, or multi-dimensional image data which may e.g. comprises three-dimensional data in the field of view 10 at different spectral wavelengths.

The camera assembly 8 may comprise at least one of an RGB camera, a multi-spectral camera and a hyper-spectral camera.

The interoperative image data comprise or consist of fluorescent-light image data. Such fluorescent-light image data may be obtained when a fluorophore 22, such as indocyanine green, is injected into the tissue 12 and illuminated at wavelengths which trigger the fluorescence.

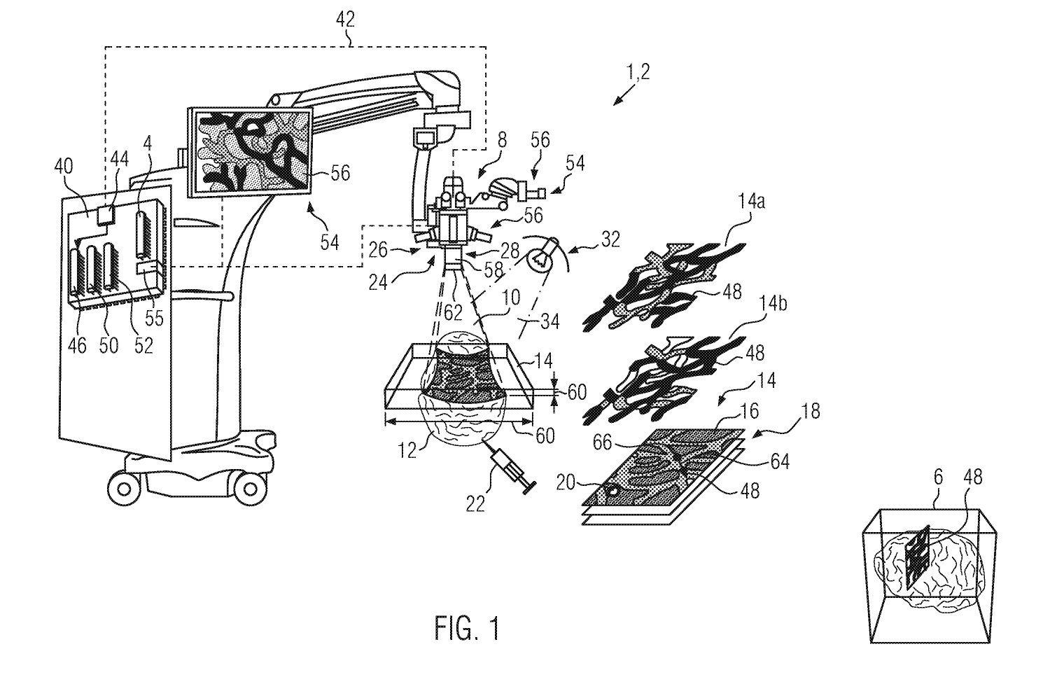

The camera assembly 8 may comprise one or more filter assemblies 24, which are only schematically shown in FIG. 1. The filter assembly 24 may comprise a filter arrangement 26 for blocking specular reflections. Examples of such a filter arrangement are described in European patent application EP 16 204 933.2, which is herewith incorporated by reference in its entirety.

The filter assembly 24 may also comprise a band-pass filter arrangement 28 for restricting the light in the interoperative image data 14 to the fluorescence wavelengths of the at least one fluorophore 22. Such a filter arrangement is shown in European patent application EP 17 179 019.8, which is herewith incorporated in its entirety by reference.

The medical observation device 1 may further include an illumination assembly 32 for generating illumination light 34 having an illumination spectrum. The illumination spectrum may be restricted to or include wavelengths that trigger fluorescence of the at least one fluorophore 22. The illumination light 34 may further comprise or be restricted to wavelengths that match the reflectance spectrum of arterial blood. The illumination light 34 may be restricted to or comprise wavelengths that are matched to the reflectance spectrum of venous blood. Restricting the illumination spectrum of the illumination light 34 to a single or to preferably separate wavelengths reduced cross-talk between the various frequency bands. This facilitates an automatic analysis of the interoperative image data 14. Subsequent input frames 16 may have been acquired at different illumination spectra. Alternatively, the interoperative image data 14 contain information preferably in at least one of the visible-light spectra, e.g. in at least one of the reflective spectrum of arterial blood and venous blood, and the near-infrared spectrum, e.g. in the fluorescence wavelengths of the at least one fluorophore 22.

The medical observation device 1 further includes an image processor assembly 40, which only by way of example is shown as an integrated circuit in FIG. 1. The image processor 40 and its constituents may be software-implemented, hardware-implemented or be implemented as a combination of hardware and software. The memory assembly 4 may be part of the image processor 40. The image processor assembly 40 may comprise several modules which may be differentiated functionally and/or structurally. The image processor assembly 40 is connected to the camera assembly 8 by a data connection 42, which may be wired and/or wireless. An input interface 44 of the image processor assembly 40 may be adapted to acquire interoperative image data 14 from at least one camera assembly 8 and/or a storage where the interoperative image data 14 are stored or buffered, e.g. after pre-processing, such as memory assembly 4.

The image processor assembly 40 may comprise a pattern-matching module 46 for identifying at least one vascular plexus structure 48 in the interoperative image data 14 and for identifying the at least one identified vascular plexus structure 48 in the pre-operative three-dimensional image data 6.

The at least one vascular plexus structure 48 may be identified e.g. in interoperative image data 14a which are restricted to the fluorescence spectrum of the fluorophore 22. In addition or alternatively, the at least one vascular plexus structure 48 may be identified in interoperative image data 14b which have been recorded in the visible-light spectrum and may in particular be restricted to at least one of the reflectance spectrum of arterial blood and venous blood. Algorithms for identifying a vascular plexus structure 48 in the interoperative image data 14 and then identifying this structure in the pre-operative three-dimensional image data 6 are, for example, given in Rouchdy, Y.; Cohen, L.: “A Geodesic Voting Method of Tubular Tree and Centrelines”, DOI: 10.1109/ISBI.2011.5872566, pp. 979-983, and Suri, J. S.; Laxminarayan, S. (eds): “Angiography and Plaque Imaging: Advanced Segmentation Techniques”, CRC Press, pp. 501-518. Further, a method of identifying vascular plexus structures by using a bolus of at least one fluorophore is described in EP 17 174 017.5, which is included in its entirety by reference.

The image processor assembly 40 may further comprise an elastic-matching module 50 for elastically matching the pre-operative three-dimensional image data 6 to the interoperative image data 14 based on the at least one identified vascular plexus structure 48. Again, an algorithm for performing such elastic matching is described in IRCS Technical Reports Series, 192, “Elastically Deforming a Three-Dimensional Atlas to Match Anatomical Brain Images” as given above. As a result of the elastic matching, the pre-operative three-dimensional image data 6 are shifted, rotated and/or distorted so that the identified vascular plexus structure 48 in both data coincides geometrically. For this, the step of elastic matching may also define a section through the pre-operative three-dimensional image data 6 which results in the field of view 10 represented in the interoperative image data 14. Further, if the camera assembly 8 comprises at least one of a multispectral camera and a hyperspectral camera for acquiring the interoperative image data 14, the blood vessel structure 48 and its type may be determined using the apparatus and method described in Opt. Express 17, 15670-15678, “Ultra-fast Multispectral Optical Imaging of Cortical Oxigenation, Blood Flow, and Intracellular Calcium Dynamics”, as given above.

The image processor assembly 40 may further comprise an image-forming module 52 for combining the elastically matched pre-operative three-dimensional image data 6 or a section thereof with the interoperative image data 14 into output image data 54. The image processor assembly may further comprise an output interface 55 for outputting the output image data 54. The output interface 55 may comprise at least one standard connector, such as an HDMI, DVI, RGB or any other suitable type of connector, and/or a wireless connection, including the matching data transmission protocol.

For displaying the output image data 54, the medical observation device 1 may comprise a display assembly 56, which may include stereoscopic display, for example an eyepiece of a microscope or endoscope and/or a monitor. The display assembly 56 may be connected to the output interface 55 by wire or wireless.

The computational burden for locating and/or identifying the identified vascular plexus structure 48 of the interoperative image data 14 in the pre-operative three-dimensional image data 6 may be high. This burden can be reduced if position information is provided as to where the field of view 10, the vascular plexus structure 48, and/or the interoperative image data 14 are located within the soft biological tissue 12. This information is useful if, for example, an elastic-matching process has already been successfully carried out for one input frame 16 of a time series 18 and then has to be repeated for subsequent input frame 16 of the time series 18 later during surgery. For this, the medical observation device 1 may comprise at least one position sensor 58 for acquiring and/or providing position data 60 representative of at least one of focal length, field depth and distance setting of an optical lens system 62 of the medical observation device 1 and size, distance from the optical lens system 62 and the image processor assembly 40, such as a dimension of the field of view 10.

The position data 60 may be input into at least one of the pattern-matching modules 46, the elastic-matching module 50 and the image-forming module 52 and be used in identifying the at least one identified vascular plexus structure 48 in the pre-operative three-dimensional image data 6, the elastic matching of the pre-operative three-dimensional image data 6 to the interoperative image data 14 and for forming the output image data 54 from the elastically matched pre-operative three-dimensional image data 6 and the interoperative image data 14.

The image processor assembly 40 may be adapted to compute the blood flow direction 64 at a location 66 in the at least one identified vascular plexus structure 48 as is described in European patent application EP 17 210 909.2 which is included by reference in their entirety. A location may be a pixel 20 or a preferably coherent array of pixels in the interoperative image data 14. The tissue type of the identified vascular plexus structure 48, i.e. a differentiation between venous, arterial and capillary tissue and/or vessels may be carried out using the method and system described in EP 17 174 047.5.

As described, the above-described system and method rely solely on information that is automatically acquired when the interoperative image data 14 are recorded. The system and method may further comprise positioning data 60 that result from the settings of the medical observation device 1 for acquiring the interoperative image data 14.

### REFERENCE NUMERALS

1 medical observation device
2 microscope
4 memory assembly
6 pre-operative three-dimensional image data
8 camera assembly
10 field of view
12 soft biological tissue
14 interoperative image data
14a fluorescent-light interoperative image data
14b visible-light interoperative image data
16 input frame
18 time series of input frames
20 pixel
22 fluorophore
24 filter assembly
26 filter arrangement for blocking specular reflections
28 band-pass filter arrangement for fluorescent light
32 illumination assembly
34 illumination light
40 image processor assembly
42 data connection
44 input interface
46 pattern-matching module
48 vascular plexus structure
50 elastic-matching module
52 image-forming module
54 output image data
55 output interface
56 display assembly
58 position sensor
60 positioning data
62 optical lens system
64 blood flow direction
66 location

What is claimed is:

1. An image processing method for displaying output image data (54) of soft biological tissue (12) in real time during surgery, comprising the steps of:
providing pre-operative three-dimensional image data (6) of the soft biological tissue (12);
acquiring interoperative image data (14) of the soft biological tissue (12) in at least one of the visible-light spectrum and the near-infrared spectrum;
automatically identifying at least one vascular plexus structure (48) in the interoperative image data (14);
identifying the at least one identified vascular plexus structure (48) in the pre-operative three-dimensional image data (6);
elastically matching the pre-operative three-dimensional image data (6) to the interoperative image data (14) based on the at least one identified vascular plexus structure (48); and
forming the output image data (54) from the elastically matched pre-operative three-dimensional image data (6) and interoperative image data (14).

2. The image processing method according to claim 1, wherein the interoperative image data (14) in which the vascular plexus structure (48) is automatically identified is acquired using fluorescent light from a fluorophore (22).

3. The image processing method according to claim 1, wherein the identified vascular plexus structure (48) in the interoperative image data (14) has been recorded using light in the visible spectrum.

4. The image processing method according to claim 1, further comprising the step of displaying the output image data (54).

5. The image processing method according to claim 1, wherein at least one optical filter arrangement (26) for reducing specular reflections is used for acquiring the interoperative image data (14).

6. The image processing method according to claim 1, wherein a blood flow direction (64) is computed at at least one location (66) in the identified vascular plexus structure (48).

7. The image processing method according to claim 4, further comprising the step of acquiring position data (60) representative of a position of a field of view (10) of an optical lens system (62) used for acquiring the interoperative image data (14) at the time of acquiring the interoperative image data (14), and wherein the position data (60) are used in at least one of identifying the vascular plexus structure (48) in the pre-operative three-dimensional image data (6), elastically matching the pre-operative three-dimensional image data (6) to the interoperative image data (14), and displaying the output image data (54).

8. The image processing method according to claim 7, wherein the position data comprise at least one of a focal length of the optical lens system (62), a field depth of the optical lens system (62), and a distance setting of the optical lens system (62).

9. The image processing method according to claim 7, wherein the position data comprise a size, a dimension and an orientation of the field of view (10) of the optical lens system (62).

10. A medical observation device (1) for generation of output image data (54) of soft biological tissue (12) during surgery, the medical observation device (1) comprising:
a memory assembly (4) comprising pre-operative three-dimensional image data (6) of the soft biological tissue (12);
a camera assembly (8) for acquiring interoperative image data (14) of the soft biological tissue (12) in at least one of the visible-light spectrum and the near-infrared spectrum;
an image processor assembly (40) which comprises
a pattern-matching module (46) configured to identify at least one vascular plexus structure (48) in the interoperative image data (14) and to identify the at least one identified vascular plexus structure (48) in the pre-operative three-dimensional image data (6),
a matching module (50) configured to elastically match the pre-operative three-dimensional image data (6) to the interoperative image data (14) based on the at least one identified vascular plexus structure (48), and
an image-forming module (52) configured to combine at least part of the elastically matched pre-operative three-dimensional image data (6) with the interoperative image data (14) to form the output image data (54); and
an output interface (55) configured to output the output image data (54).

11. The medical observation device (1) according to claim 10, further comprising a display assembly (56) for displaying the output image data (54).

12. The medical observation device (1) according to claim 10, wherein the camera assembly (8) comprises at least one filter arrangement (26) for reducing specular reflections, the filter arrangement (26) comprising at least one pair of cross-polarizers.

13. The medical observation device (1) according to claim 10, wherein the camera assembly (8) comprises at least one filter arrangement (28) having a pass band matched to a fluorescence spectrum of at least one fluorophore (22).

14. The medical observation device (1) according to claim 10, wherein the camera assembly (8) comprises an optical lens system (62) and at least one position sensor (58) for acquiring position (60), the position data (60) being representative of at least one of a focal length, a field depth and a distance setting of the optical lens system (62), or a size, dimension and orientation of the field of view (10).

15. The medical observation device (1) according to claim 10, wherein the image processor assembly (40) is configured to compute a blood flow direction (64) in the identified vascular plexus structure (48) and wherein the image-forming module (52) is adapted to combine the identified vascular plexus structure (48) in the output image data (54) with time-varying marker data which are representative of the blood flow direction (64).

16. The medical observation device (1) according to claim 10, wherein the medical observation device (1) is a microscope (2).

17. The medical observation device (1) according to claim 10, wherein the medical observation device (1) is an endoscope.

18. A non-transitory computer-readable medium storing a programme causing a computer to execute the image processing method according to claim 1.
